(19) United States
(12) Patent Application Publication
(10) Pub. No.: US 2007/0088439 A1
(43) Pub. Date: Apr. 19, 2007

(54) ARTIFICIAL DISC WITH ENDPLATES HAVING CAGES TO PROMOTE BONE FUSION

(76) Inventor: Jeffery Thramann, Longmont, CO (US)

Correspondence Address: HOLLAND & HART, LLP, P.O BOX 8749, DENVER, CO, 80201, US

(21) Appl. No.: 11/163,299

(22) Filed: Oct. 13, 2005

(51) Int. Cl. A61F 2/44

(52) U.S. Cl. 623/17.13; 623/17.11

(57) ABSTRACT

The present invention provides an anchor to attach an artificial disc to vertebral bodies. The artificial disc comprises a superior endplate and an inferior endplate. A disc section extends between the endplates. The superior endplate comprises a first wall defining a first cavity, the first cavity to contain material to facilitate fusion of the superior endplate to a superior vertebral body. Similarly, the inferior endplate comprises a second wall defining a second cavity, the second cavity to contain material to facilitate fusion of the superior endplate to a superior vertebral body.

### FIELD OF THE INVENTION

The present invention relates to spinal implants and, more particularly, to an artificial disc including endplates having cages to promote bone growth to facilitate attaching the endplates to the inferior face of the superior vertebral body and the superior face of the inferior vertebral body.

### BACKGROUND OF THE INVENTION

For a number of years, surgical spinal correction has been tending away from conventional fusion surgical technologies to non-fusion technologies. With fusion technology, a graft is placed between superior and inferior vertebrae. Bone growth is encouraged to fuse the superior vertebrae and the inferior vertebrae. Once fused, the pain is frequently removed or reduced, but fusion is a less than satisfactory solution because of the resulting limitation on the range of motion caused by one or more fusions. Moreover, fusing vertebrae increases the stress on adjacent levels.

Non-fusion technologies, while providing a greater range of motion and the like, have a number of problems also. In some attempts at disc replacement, for example, a flexible artificial disc is placed within the intervertebral disc space. In these systems, the expectation is the disc will remain in place based on endplate fibrous and bone in growth and/or axial loading of the cervical spine. Often, these types of discs fail as they migrate out of the disc space. They also present a potential danger for a stretch injury to the spinal cord due to minimally restrained in extension.

Thus, it would be beneficial to design an artificial disc that provides a better anchor to the vertebral bodies.

### SUMMARY OF THE INVENTION

The present invention provides an anchor to attach an artificial disc to vertebral bodies. The artificial disc comprises a superior endplate and an inferior endplate. A disc section extends between the endplates. The superior endplate comprises a first wall defining a first cavity, the first cavity to contain material to facilitate fusion of the superior endplate to a superior vertebral body. Similarly, the inferior endplate comprises a second wall defining a second cavity, the second cavity to contain material to facilitate fusion of the superior endplate to a superior vertebral body.

The foregoing and other features, utilities and advantages of the invention will be apparent from the following more particular description of a preferred embodiment of the invention as illustrated in the accompanying drawings.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a view of an artificial disc consistent with an embodiment of the present invention;

FIG. 2 shows a cross-sectional view of the superior endplate of FIG. 1;

FIG. 3 shows another cross-sectional view of the superior endplate of FIG. 1;

FIG. 4 shows alternative internal walls shown in FIG. 2; and

FIG. 5 shows a view of the artificial disc of FIG. 1 in a spine with a stabilization device to facilitate fusion of the endplates.

### DETAILED DESCRIPTION

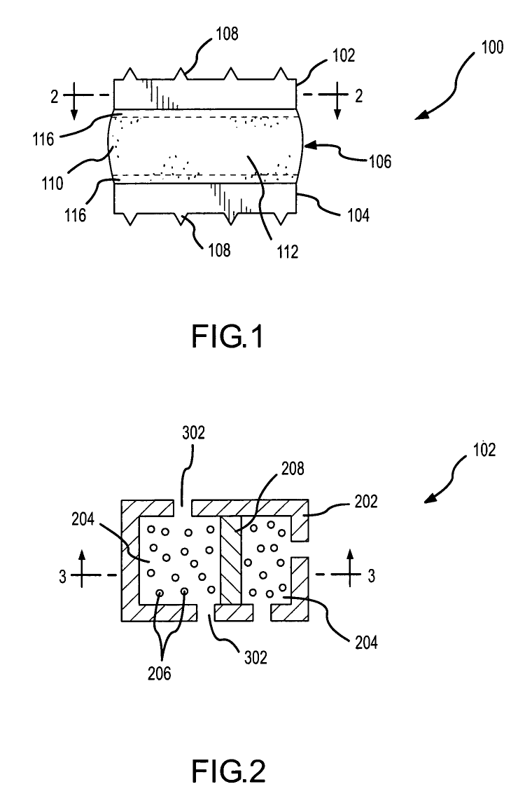

Referring to FIG. 1, an artificial disc 100 is shown. Artificial disc 100 includes a superior endplate 102, an inferior endplate 104, and a disc section 106. Superior endplate 102 and inferior endplate 104 may have protrusions 108 to facilitate implanting artificial disc 100. Protrusions 108 may be bone spikes, ridges, teeth, striations or the like. Disc section 106 may be protected by a cover 110.

Disc section 106 comprises an elastic material 112 in the disc portion of disc section 106 designed to facilitate artificial disc 100 mimicking the anatomical disc it is replacing. Elastic material 112 in disc section 106 could be a gel, a resin, a spring, shaped memory material, such as NiTi, or the like. Some artificial discs having elastic materials are described in, for example, U.S. Pat. No. 6,770,094, titled INTERVERTEBRAL DISC PROSTHESIS, issued Aug. 3, 2004, to Fehling et al., incorporated herein by reference as if set out in full, U.S. patent application Ser. No. 10/641,530, titled SHAPED MEMORY ARTIFICIAL DISC AND METHODS OF ENGRAFTING THE SAME, filed Aug. 14, 2004, incorporated herein by reference as if set out in full, and a U.S. Pat. No. 6,881,228, titled ARTIFICIAL DISC IMPLANT, issued Apr. 19, 2005, to Zdeblick et al., incorporated herein by reference as if set out in full. Of course, as one of ordinary skill in the art will recognize on reading the disclosure, reference to the particular types of artificial discs described is provided as exemplary and should not be considered limiting.

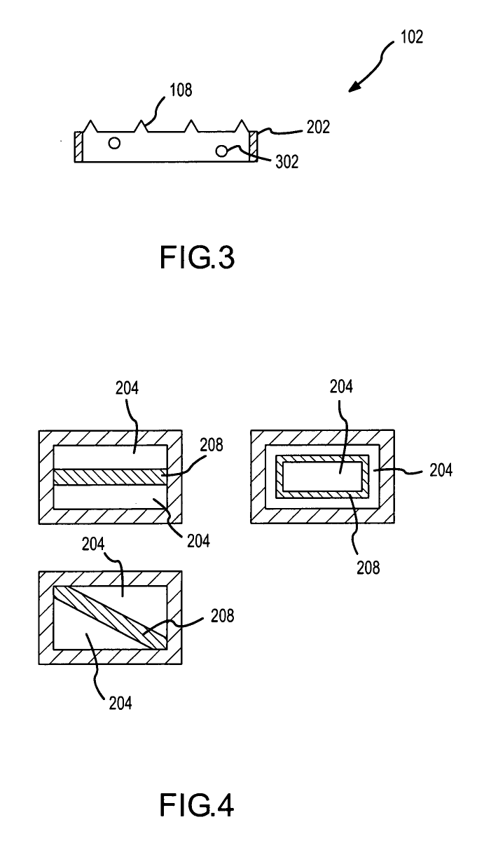

Referring now to FIGS. 2 and 3, cross-sectional views of superior endplate 102 are provided. Inferior endplate 104 would be similar and is not provided for convenience. Superior endplate 102 comprises at least one wall 202 defining an interior cavity 204. Wall 202 may be contiguous or noncontiguous. Optionally, wall 202 may have perforations 302 or holes. Perforations 302 provide access points for bone growth into superior endplate 102. Cavity 204 may contain bone growth material 206, such as, for example, cancellous bone, osteogenic material, and the like.

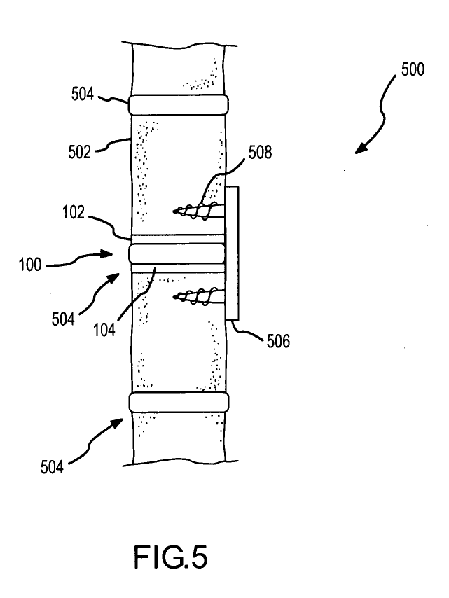

Optionally, cavity 204 may have one or more internal wall 208. Internal wall 208 may be arranged to along the long dimension, short dimension, diagonal, or the like of superior endplate 102. FIG. 4 shows alternative shapes for internal wall 208.

As shown, superior endplate 102 and inferior endplate are rectangular in shape. However, alternative shapes are possible. Other shapes include circular, elliptical, square, other polygon shapes, or random shapes. The shape may be dictated by the anatomy of the spinal to which the artificial disc is going to be implanted.

While a particular design for superior endplate 102 (and inferior endplate 104) has been shown, one of skill in the art will now recognize superior endplate 102 may have a number of similar designs. In particular, superior endplate 102 and inferior endplate 104 may be constructed similar to conventional bone fusion cages, such as, for example, U.S. Pat. No. 6,902,581, titled APPARATUS FOR FUSING ADJACENT BONE STRUCTURE, issued Jun. 7, 2005, to. Walkenhorst, et al., incorporated herein by reference as if set out in full.

Bone growth and fusion of superior endplate 102 with the inferior surface of the superior vertebrae and inferior endplate 104 with the superior surface of the inferior vertebrae facilitates anchoring artificial disc 100. The anchoring should inhibit artificial disc from moving and decrease the failure rates of the associated artificial disc.

While bone growth is desired around endplates 102 and 104, bone growth is not desired about disc section 106. Thus, disc section 106 may comprise cover 110 around the elastic portion. Cover 110 could be made from material that inhibits bone growth. Moreover, a barrier 116 may be placed between superior endplate 102 and disc section 106, and between inferior endplate 104 and disc section 106. Because disc section 106 extends and flexes with the movement of the back, it is unlikely bone would grow or fuse to disc section 106; therefore, having cover 110 and barriers 112 that inhibit bone growth into the disc section 106 is optional.

Referring now to FIG. 5, a portion of a spine 500 is shown. Spine 500 comprises a series of vertebral bodies 502 and disc spaces 504. Artificial disc 100 occupies at least one of the disc spaces 504 with superior endplate 102 attached to one vertebral body 502 and inferior endplate 104 attached to another vertebral body 502. Also, shown in FIG. 500 a stabilization device 506, which is shown as a conventional cervical plate or the like however, stabilization device can be any conventional device and is not necessarily a plate, is placed to stabilize the vertebral bodies 502 to facilitate bone growth into endplates 102 and 104. Stabilization device 506 would be attached to the vertebral bodies 502 using anchors 508, anchors 508 may be any conventional mechanism for attaching stabilization device 506, such as, for example, pedicle screws, clamps, or the like. Stabilization device 506 provides a mechanism to stabilize vertebral bodies 502 and artificial disc 100 to facilitate bone growth into endplates 102 and 104. Of course, one of skill in the art would recognize stabilization device 506 would limit the range of motion of the spine and potentially encourage fusion of vertebral bodies 502 around artificial disc 100. Thus, stabilization device 506 and the anchors 508 should comprise resorbable material.

While the invention has been particularly shown and described with reference to an embodiment thereof, it will be understood by those skilled in the art that various other changes in the form and details may be made without departing from the spirit and scope of the invention.

What is claimed is:

1. An artificial disc comprising:
a superior endplate;
an inferior endplate; and
a disc section extending between the superior endplate and the inferior endplate, wherein the superior endplate comprises a first wall defining a first cavity, the first cavity to contain material to facilitate fusion of the superior endplate to a superior vertebral body, and the inferior endplate comprises a second wall defining a second cavity, the second cavity to contain material to facilitate fusion of the superior endplate to a superior vertebral body.

2. The artificial disc of claim 1, wherein the material to facilitate fusion comprises cancellous bone.

3. The artificial disc of claim 1, wherein the material to facilitate fusion comprises osteogenic material.

4. The artificial disc of claim 1, wherein the superior endplate comprises at least one superior perforation in the first wall.

5. The artificial disc of claim 4, wherein the inferior endplate comprises at least one inferior perforation in the second wall.

6. The artificial disc of claim 1, wherein the superior endplate comprises a fusion cage structure.

7. The artificial disc of claim 6, wherein the inferior endplate comprises a fusion cage structure.

8. The artificial disc of claim 1, wherein the disc section comprises a cover.

9. The artificial disc of claim 8, wherein the cover inhibits bone growth.

10. The artificial disc of claim 1, further comprising a superior bone growth barrier between the superior endplate and the disc section and an inferior bone growth barrier between the inferior endplate and the disc section.

11. The artificial disc of claim 1, further comprising at least one superior protrusion on the superior endplate to facilitate anchoring with the superior vertebral body and at least one inferior protrusion on the inferior endplate to facilitate anchoring with the inferior vertebral body.

12. The artificial disc of claim 11, wherein the at least one superior protrusion and the at least one inferior protrusion comprise a spike.

13. The artificial disc of claim 1, further comprising a stabilization device to stabilize the spine and facilitate fusion of the superior endplate and the inferior endplate.

14. The artificial disc of claim 13, wherein the stabilization device comprises a resorbable plate coupled to the superior vertebral body and the inferior vertebral body.

15. An artificial disc comprising:
a superior endplate;
an inferior endplate; and
an elastic material residing between the superior endplate and the inferior endplate, wherein
the superior endplate comprises at least one superior perforation to facilitate bone growth between a superior vertebral body and the superior endplate, and
the inferior endplate comprises at least one inferior perforation to facilitate bone growth between the inferior vertebral body and the inferior endplate.

16. The artificial disc of claim 15, wherein
the superior endplate comprises a first sidewall defining a first cavity and wherein the at least one superior perforation resides in the first sidewall;
the inferior endplate comprises a second sidewall defining a second cavity and wherein the at least one inferior perforation resides in the second sidewall.

17. The artificial disc of claim 16, wherein the at least one superior perforation comprises a plurality of holes and the at least one inferior perforation comprises a plurality of holes such that the superior endplate and the inferior endplate function as a bone fusion cage.

18. The artificial disc of claim 16, further comprising bone growth material packed into the first cavity and the second cavity.

19. The artificial disc of claim 18, wherein the elastic material is encased by a cover that inhibits bone growth.

20. The artificial disc of claim 19, wherein the elastic material comprises at least a shaped memory alloy.

21. The artificial disc of claim 17, wherein further comprising a first bone growth barrier between the superior endplate and the superior vertebral body and a second bone growth barrier between the inferior endplate and the inferior vertebral body.

22. An artificial disc comprising:
a superior endplate;
an inferior endplate; and
an elastic material residing between the superior endplate and the inferior endplate, wherein
the superior endplate comprises at least one superior bone fusion cage to facilitate bone growth between a superior vertebral body and the superior endplate, and
the inferior endplate comprises at least one inferior bone fusion cage to facilitate bone growth between the inferior vertebral body and the inferior endplate.

23. The artificial disc according to claim 22, further comprising packing at least one of the superior bone fusion cage and the inferior bone fusion cage with bone growth material.

24. The artificial disc according to claim 22, further comprising a stabilization device extending between the superior vertebral body and the inferior vertebral body to facilitate bone growth in the at least one superior bone fusion cage and the at least one inferior bone fusion cage.

25. The artificial disc according to claim 24, wherein the stabilization device comprises a plate attached to the superior vertebral body and the inferior vertebral body using pedicle screws.

26. The artificial disc according to claim 24, wherein the stabilization device is resorbable.
